(19) United States
(12) Patent Application Publication
(10) Pub. No.: US 2024/0366145 A1
(43) Pub. Date: Nov. 7, 2024

(54) SYSTEMS, DEVICES, AND METHODS FOR THERMAL IMAGING

(71) Applicant: MOLECULIGHT INC., Toronto (CA)

(72) Inventors: Ralph S. DaCosta, Etobicoke (CA); Garry MORGAN, Mississauga (CA); Samuel J. SALEMI, Toronto (CA); Danielle Dunham, Scarborough (CA); Nitesh MISTRY, Toronto (CA)

(73) Assignee: MOLECULIGHT INC., Toronto (CA)

(21) Appl. No.: 18/647,718

(22) Filed: Apr. 26, 2024

Related U.S. Application Data
(60) Provisional application No. 63/498,516, filed on Apr. 26, 2023.

(51) Int. Cl. A61B 5/00; A61B 5/01; G06T 7/00

(52) U.S. Cl. CPC A61B 5/445; A61B 5/0035; A61B 5/0071; A61B 5/0077; A61B 5/015; A61B 5/7425; A61B 5/748; G06T 7/0014; G06T 2207/10048; G06T 2207/10064; G06T 2207/30088

(57) ABSTRACT

Systems, devices, and methods for thermal imaging of a wound are provided. A multi-modal imaging device is configured to capture fluorescence images and thermal images of a target such as a wound. The device is further configured to determine a temperature differential between two user-selected regions of one or more of the captured images and provide an output indicative of the temperature differential. The output display also includes the fluorescence image.

### CROSS REFERENCE TO RELATED APPLICATIONS

The present Application claims priority to U.S. Provisional Application No. 63/498,516, filed in the United States Patent and Trademark Office on Apr. 26, 2023, the entire contents of which are incorporated by reference herein.

### TECHNICAL FIELD

The present disclosure relates to methods, systems and devices for fluorescent and thermal imaging of a target such as a wound.

### BACKGROUND

Wound care is a major clinical challenge. Healing and chronic non-healing wounds are associated with a number of biological tissue changes including inflammation, necrosis, production of exudate, bleeding, proliferation, remodeling of connective tissues, and a common major concern, bacterial presence, growth and infection. A portion of wound infections are not clinically apparent and contribute to the growing personal, emotional, and economic burdens associated with wound care, especially in aging populations. For example, Pseudomonas aeruginosa and Staphylococcus aureus are genera of bacteria that are prevalent in hospital settings and are common causes of bacterial infection. Currently, the clinical gold standard of wound assessment includes direct visual inspection of the wound site under white light illumination for classical signs and symptoms of infection. This is often combined with a swab culture or tissue biopsy sample for laboratory testing.

Certain medical specialties (e.g., cardiology, oncology, neurology, orthopedics, etc.) rely on particular imaging modalities (e.g., x-ray, ultrasound, magnetic resonance imaging (MRI), computed tomography (CT) scans, etc.) to assist with the diagnosis and assessment. Clinicians in such specialties may use advanced and established methods of interpreting the images. In wound care specialties, by contrast, the standard of care has not historically relied on such imaging modalities and no such advanced or established methods of interpreting the images exist. While some clinicians may use cameras to capture images of a wound in a standard photographic format, these formats do not identify or expose any bacterial information within the wound.

Qualitative and subjective visual assessment only provides a gross view of the wound site, but does not provide information about underlying biological, biochemical, and molecular changes that are occurring at the tissue and cellular level. Moreover, bacteria are invisible to the unaided eye, resulting in suboptimal wound sampling and an inability to appropriately track changes in bacterial growth in the wound site. This can impede healing and timely selection of the optimum anti-microbial treatment. Moreover, it may be difficult to differentiate certain markers of bacterial presence from similar markers caused by non-bacterial sources. For example, a fluorescence image may contain reflections from non-bacterial sources (e.g., tattoos, fingernails, toenails, jewelry, background environment, etc.) which appear to be similar in color to the fluorescence that would be expected from certain strains of bacteria. Further, while fluorescence images may indicate the presence of bacteria in or around a wound, the presence of bacteria does not necessarily indicate the presence of infection and similarly, the lack of bacterial presence does not necessarily indicate that a wound is healed. Stopping antibiotics before an infection is healed may delay healing and providing antibiotics that are no longer (or are not) needed may result in unnecessary treatment or potentially delay a different type of treatment.

Therefore, there exists a need for systems, devices, and methods for the capturing of medical images (such as fluorescence images of wound) which may reliably indicate whether the presence of bacteria represents an infection, whether a reduced bacterial load indicates a wound has healed or an infection has resolved, may help to identify newly forming wounds, and may also provide information as to why a wound is slow to heal, thus reducing both morbidity and mortality due to wounds.

### SUMMARY

The present disclosure may demonstrate one or more of the above-mentioned desirable features. Other features and/or advantages may become apparent from the description that follows.

In accordance with one aspect of the present disclosure, a method of analyzing a wound is provided. The method includes capturing a first image of a target and a surrounding area with a handheld multi-modal imaging device, capturing a second image of the target and the surrounding area with the handheld multi-modal imaging device, wherein the second image is a thermal image. The method further includes co-registering the first image and the second image, receiving a first input from a user of the handheld multi-modal imaging device, the first input identifying a reference point on the target and the surrounding area, and receiving a second input from the user of the handheld multi-modal imaging device, the second input identifying a test point on the target and the surrounding area. The method also includes determining, with a processor of the multi-modal imaging device, a difference in temperature between the reference point and the test point and outputting, to a display of the multi-modal imaging device, an indication of the difference in temperature and the co-registered images.

In accordance with another aspect of the present disclosure, a multi-modal imaging device is provided. The imaging device includes an optical sensor configured to detect fluorescence from a target and a surrounding area, a thermal imaging module configured to capture a thermal image of the target and the surrounding area, a display and a processor. The processor is configured to co-register a fluorescence image and a thermal image, receive a first input from a user of the device, the first input identifying a reference point on the target and the surrounding area, receive a second input from the user of the device, the second input identifying a test point on the target and the surrounding area, determine a difference in temperature between the reference point and the test point, and output an indication of the difference in temperature to the display.

In accordance with yet another aspect of the present disclosure, a method of analyzing a wound includes capturing a white light image of a target and a surrounding area with a handheld multi-modal imaging device, capturing a fluorescence image of the target and the surrounding area with the handheld multi-modal imaging device, and capturing a thermal image of the target and the surrounding area with a handheld multi-modal imaging device. The method further discloses selecting a reference area on one of the white light image, the fluorescence image, and the thermal image on a display of the handheld multi-modal imaging device and selecting a test area on the one of the white light image, the fluorescence image, and the thermal image on the display of the handheld multi-modal imaging device. The method also includes determining, with a processor of the multi-modal imaging device, a difference in temperature between the reference area and the test area, and outputting to the display an indication of the difference in temperature.

In accordance with yet another aspect of the present disclosure, a method of analyzing a wound includes capturing a fluorescence image of a target and a surrounding area with a handheld multi-modal imaging device and capturing a thermal image of the target and the surrounding area with the handheld multi-modal imaging device. The method further discloses selecting, on one of the captured images, a reference area and a test area using a display of the handheld multi-modal imaging device. The method also includes determining, with a processor of the multi-modal imaging device, a difference in temperature between the reference area and the test area, and outputting to the display the fluorescence image, the thermal image, and an indication of the difference in temperature.

In accordance with a further aspect of the present disclosure, a multi-modal imaging device includes a housing, a white light camera configured to capture white light images, a fluorescence camera configured to capture fluorescence images, a thermal imaging module, a processor configured to receive white light image data, fluorescence image data, and thermal image data, and a display. The display is configured to allow a user to select a reference area and a test area on one or more of a white light image, a fluorescent image, and a thermal image. The processor is further configured to determine a difference in temperature between the reference area and the test area, and to output to the display an indication of the difference in temperature.

In accordance with yet another aspect of the present disclosure, a multi-modal imaging device includes a housing, a fluorescence camera configured to capture fluorescence images, a thermal imaging module, a processor configured to receive fluorescence image data and thermal image data, and a touchscreen display. The display is configured to allow a user to select a reference area and a test area on a captured image, and the processor is further configured to determine a difference in temperature between the user-selected reference area and the user-selected test area. The processor outputs to the display a fluorescence image containing the reference area and the test area and an image co-registered with the fluorescence image and containing an indication of the difference in temperature between the user-selected reference area and the user-selected test area.

In accordance with another aspect of the present disclosure, a method of analyzing a wound includes capturing a fluorescence image of a target and a surrounding area with a handheld multi-modal imaging device and capturing a thermal image of the target and the surrounding area with the handheld multi-modal imaging device. The method further includes selecting, via a touchscreen display of the handheld multi-modal imaging device, on one of the captured images, a reference area and a test area, and determining, with a processor of the multi-modal imaging device, a difference in temperature between the reference area and the test area. The method also includes displaying, on the touchscreen display of the handheld multi-modal imaging device, the fluorescence image and the thermal image, and an indication of the difference in temperature between the reference area and the test area.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be understood from the following detailed description either alone or together with the accompanying drawings. The drawings are included to provide a further understanding of the disclosed teachings and are incorporated in and constitute a part of this specification. The drawings illustrate one or more example embodiments of the present disclosure and together with the description serve to explain various principles and operations.

FIG. 1A is a rear perspective view of an example embodiment of a multi-modal imaging device without a thermal module attached, in accordance with the present disclosure.

FIG. 1B is a front perspective view of the multi-modal imaging device of FIG. 1A without the thermal module attached.

FIG. 2A is a perspective side view of a second example embodiment of a multi-modal imaging device with a thermal module attached in accordance with the present disclosure.

FIG. 2B is a front side perspective view of the multi-modal imaging device of FIG. 2A with the thermal module attached.

FIG. 2C is another front perspective view of the multi-modal imaging device of FIGS. 2A-2B with a thermal module attached.

FIG. 2D is a rear perspective view of the multi-modal imaging device of FIGS. 2A-2C without the thermal module attached and showing a display screen of the multi-modal imaging device.

FIG. 3 is a flowchart illustrating an example method of using the multi-modal imaging device of the present disclosure to analyze a target.

FIG. 4A is an example relative color scale showing colors representing a difference in temperature between a reference point and a test point selected by a user on an image captured with the multi-modal imaging device of the present disclosure. As shown in FIG. 4A, each colored block on the scale represents an increase or decrease of one degree from the block below it or above it, respectively. Looking at the scale in FIG. 4A, the white box at the top of the scale represents a positive temperature differential of 6 degrees or more in Celsius (the test point is warmer than the reference point when the temperature differential is positive), the red box immediately below the white box represents a positive temperature differential of 5 degrees, the dark orange box immediately below the red box represents a positive temperature differential of 4 degrees, the light orange box immediately below the dark orange box represents a positive temperature differential of 3 degrees, the yellow box immediately below the light orange box represents a positive temperature differential of 2 degrees, the dark yellow/gold box immediately below the yellow box represents a positive temperature differential of 1 degree, the light green box represents a 0 degree temperature difference-this is the user selected reference point, the darker green box immediately below the light green box represents a negative temperature differential of 1 degree, the aqua box immediately below the darker green box represents a negative temperature differential of 2 degrees, the light blue box immediately below the aqua box represents a negative temperature differential of 3 degrees, the royal blue box immediately below the light blue box represents a negative temperature differential of 4 degrees, the dark blue box immediately below the royal blue box represents a negative temperature differential of 5 degrees, and the black box forming the end of the relative color scale and immediately below the dark blue box represents a negative temperature differential of 6 or more degrees relative to the user selected reference point.

FIG. 4B is an example of an alternative embodiment of a relative color scale showing colors representing a difference in temperature between a reference point and a test point selected by a user on an image captured with the multi-modal imaging device of the present disclosure. As shown in FIG. 4B, each colored block on the scale represents an increase or decrease of two degrees Celsius from the block below it or above it, respectively. Looking at the scale in FIG. 4B, the white box at the top of the scale represents a positive temperature differential of 8 degrees or more in Celsius (the test point is warmer than the reference point when the temperature differential is positive), the red box immediately below the white box represents a positive temperature differential of 6 degrees, the orange box immediately below the red box represents a positive temperature differential of 4 degrees, the yellow box immediately below the orange box represents a positive temperature differential of 2 degrees, the light green box represents a 0 degree temperature difference-this is the user selected reference point, the darker green box immediately below the light green box represents a negative temperature differential of 2 degrees, the aqua box immediately below the darker green box represents a negative temperature differential of 4 degrees, the light blue box immediately below the aqua box represents a negative temperature differential of 6 degrees, the royal blue box immediately below the light blue box represents a negative temperature differential of 8 degrees, the dark blue box immediately below the royal blue box represents a negative temperature differential of 10 degrees, and the black box forming the end of the relative color scale and immediately below the dark blue box represents a negative temperature differential of 12 or more degrees relative to the user selected reference point.

FIG. 5A shows, from left to right, co-registered standard and thermal images in which a right foot of a patient having a diabetic foot ulcer (DFU), which has healed, is 8.5° C. hotter than the contralateral limb.

FIG. 5B shows, from left to right, co-registered standard, fluorescent, and thermal images of the underside of the hotter foot in FIG. 5A. As can be seen in the images, a new wound was found on the big toe of the right foot, which when compared to the other toes of the same foot, was found to be 5.1° C. hotter.

FIG. 6 shows, from left to right, co-registered standard, fluorescent, and thermal images of a sacral pressure ulcer. As can be seen in the images, the wound was negative for fluorescence, ruling out a bacterial infection, but the thermal image identified a cool region, leading to the identification of extensive tunneling and undermining.

FIG. 7 shows, from left to right, co-registered standard, fluorescence, and thermal images of a wound (deep pilonidal cyst). As can be seen in the images, the fluorescent image indicates heavy bacterial load (see white arrow) and the thermal image shows cool regions overlapping with the FL positive areas, indicating poor perfusion and devitalization of tissue.

FIGS. 8A and 8B, show images of a patient wound taken two weeks apart. Two fluorescent images are shown, the first of which was positive for bacteria and the second of which, taken two weeks later, shows resolution of surface bacteria. However, the thermal image of FIG. 8B shows that although the surface bacteria had resolved, the infection remained, as indicated by a positive 5.8° C. temperature differential between the affected and contralateral limb.

FIG. 9 shows, from top to bottom, a standard image, a fluorescence image, and a thermal image of a wound. Cooler temperatures surround the wound in areas covered by intact skin.

FIG. 10 shows, from top to bottom, a standard image, a fluorescence image, and a thermal image of an infected, chronic post-radiation skin affliction.

FIG. 11 shows, from top to bottom, a standard image, a fluorescence image, a close-up thermal image, and a distanced thermal image of a wound.

FIG. 12A shows a standard image of a limb having two wounds, wound A and wound B, and a co-registered thermal image of the same view of wound A and wound B.

FIG. 12B shows, from left to right, co-registered images of wound A of FIG. 12A, including a standard image, a fluorescence image showing red fluorescence in the wound area, and a thermal image.

FIG. 12C shows, from left to right, co-registered images of wound B of FIG. 12B, including a standard image, a fluorescence image negative for bacterial fluorescence, and a thermal image.

FIG. 13 shows, from top to bottom, a standard image, a fluorescence image, an up-close thermal image, and a distant thermal image of a pressure injury.

FIG. 14A-14C shows an example user interface (UI) for imaging, review, and analysis on a display of a monitoring device in accordance with the present disclosure.

### DESCRIPTION OF VARIOUS EXAMPLE EMBODIMENTS

Accurate and clinically relevant wound assessment is an important clinical tool, but this process currently remains a substantial challenge. Current visual assessment in clinical practice only provides a gross view of the wound site (e.g., presence of purulent material and crusting). Current best clinical practice fails to adequately use the critically important objective information about underlying key biological changes that are occurring at the tissue and cellular level (e.g., contamination, colonization, infection, matrix remodeling, inflammation, bacterial/microbial infection, and necrosis) since such indices are i) not easily available at the time of the wound examination and ii) they are not currently integrated into the conventional wound management process. Direct visual assessment of wound health status using white light relies on detection of color and topographical/textural changes in and around the wound, and thus may be incapable and unreliable in detecting subtle changes in tissue remodeling. More importantly, direct visual assessment of wounds often fails to detect the presence of bacterial infection, since bacteria are occult under white light illumination. Infection is diagnosed clinically with microbiological tests used to identify organisms and their antibiotic susceptibility. Although the physical indications of bacterial infection can be readily observed in most wounds using white light (e.g., purulent exudate, crusting, swelling, erythema), this is often significantly delayed, and the patient is already at increased risk of morbidity (and other complications associated with infection) and mortality. Therefore, standard white light direct visualization fails to detect the early presence of the bacteria themselves or identify the types of bacteria within the wound.

Wound progression is currently monitored manually. The National Pressure Ulcer Advisory Panel (NPUAP) developed the Pressure Ulcer Scale for Healing (PUSH) tool that outlines a five-step method of characterizing pressure ulcers. This tool uses three parameters to determine a quantitative score that is then used to monitor the pressure ulcer over time. The qualitative parameters include wound dimensions, tissue type, and the amount of exudate or discharge, and thermal readings present after the dressing is removed. A wound can be further characterized by its odor and color. Such an assessment of wounds currently does not include critical biological and molecular information about the wound. Therefore, all descriptions of wounds are somewhat subjective and noted by hand by either the attending physician or the nurse.

In accordance with the present teachings, methods of analysis for data collected from a wound are provided. For example, the collection of fluorescence image data has been shown to improve clinical wound assessment and management. When excited by short wavelength light (e.g., ultraviolet or short visible wavelengths), most endogenous biological components of tissues (e.g., connective tissues such collagen and elastins, metabolic co-enzymes, proteins, etc.) produce fluorescence of a longer wavelength, in the ultraviolet, visible, near-infrared and infrared wavelength ranges.

Tissue autofluorescence imaging provides a unique means of obtaining biologically relevant information of normal and diseased tissues in real-time, thus allowing differentiation between normal and diseased tissue states, as well as the volume of the diseased tissue. This is based, in part, on the inherently different light-tissue interactions (e.g., abosption and scattering of light) that occur at the bulk tissue and cellular levels, changes in the tissue morphology and alterations in the blood content of the tissues. In tissues, blood is a major light absorbing tissue component (i.e., a chromophore). This type of technology is suited for imaging disease in hollow organs (e.g., GI tract, oral cavity, lungs, bladder) or exposed tissue surfaces (e.g., skin). An autofluorescence imaging device in accordance with the present disclosure may collect wound data that provides/allows rapid, non-invasive and non-contact real-time analysis of wounds and their composition and components, to detect and exploit the rich biological information of the wound to improve clinical care and management.

Devices that capture fluorescence images of wounds and enable identification of bacterial presence, bacterial location, and bacterial load are disclosed in U.S. Pat. No. 9,042,967 B2 to DaCosta et al., entitled “Device and Method for Wound Imaging and Monitoring,” and issued on May 26, 2015. This patent claims priority to PCT Application No. PCT/CA2009/000680 filed on May 20, 2009, and to U.S. Provisional Patent Application No. 61/054,780, filed on May 20, 2008. The entire content of each of these above-identified patents, patent applications, and patent application publications is incorporated herein by reference. These documents disclose at least some aspects of a device configured to collect data for objectively assessing wounds for changes at the biological, biochemical and cellular levels and for rapidly, sensitively and non-invasively detecting the earliest presence of bacteria/microorganisms within wounds.

Additional exemplary wound monitoring devices described herein including include hand-held/portable optical digital imaging devices having specific excitation light sources and optical filters (e.g., low-pass filters, high-pass filters, band-pass filters, multi-band filters, polarization filters, etc.) attached thereto, although in some implementations the hand-held/portable optical digital imaging devices described herein may be filterless. Such exemplary devices may be configured with optical heads to permit multiple different use cases, including but not limited to endoscopic imaging, and in some implementations such devices may be modular. These devices include but are not limited to those described in provisional patent application No. 63/482,892 entitled “Systems, Devices, and Methods for Fluorescence Imaging with Imaging Parameter Modulation” filed Feb. 2, 2023, International Patent Application Publication No. WO 2016/011534 A1, entitled “Collection and Analysis of Data for Diagnostic Purposes,” and which claims priority to U.S. Provisional Patent Application No. 62/028,386, filed on Jul. 24, 2014, and International Patent Application Publication WO 2020/148726 A1, entitled “Modular System for Multi-Modal Imaging and Analysis,” and which claims priority to U.S. Provisional Patent Application No. 62/793,842 filed Jan. 17, 2019. The entire content of each of these above-identified patent applications, and patent application publications is incorporated herein by reference. Using imaging devices and systems further described herein, fluorescence of components in a wound due to exposure to excitation light may be imaged and analyzed. For example, in a wound having a bacterial presence caused by or containing, for example, Pseudomonas aeruginosa, the Pseudomonas aeruginosa fluoresce with a specific spectral signature, i.e., one or more bands of wavelengths with known peaks, when subjected to excitation light. The excitation light may comprise any light with known wavelength or range of wavelengths with known peaks, such as a peak at 405 nm. Capturing and analyzing this data permits identification of bacterial presence in general, and identification of the presence of specific types of bacteria as well. In order to identify, type, and quantify the bacterial presence as well as additional characteristics of the wound, the devices and systems are trained.

The above identified example devices also disclose the use of thermal imaging in combination with fluorescence imaging. The addition of thermal imaging in combination with fluorescence imaging provides a deeper understanding of the fluorescence emitted by wound components and bacteria within a wound. Thermal mapping of a wound can provide additional information regarding pathophysiological changes to further guide assessment and treatment of wounds.

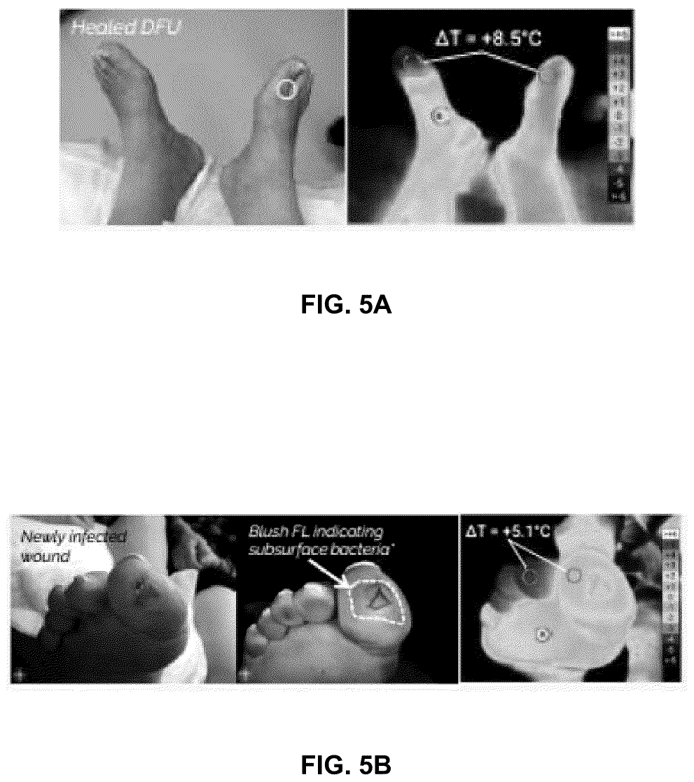

In accordance with one example embodiment, a device in accordance with the present disclosure is configured to capture white light (WL) or standard images, fluorescence images, and thermal images of a target such as as wound. A device in accordance with the present disclosure also enables co-registration and co-localization of white light/standard, fluorescence, and thermal images. A device in accordance with the present disclosure provides real-time, dynamic live thermal imaging of the target. A device in accordance with the present disclosure also auto-calculates a temperature difference, AT, between two user-selected regions of the imaged target. For example, a user may view a fluorescence image of the target and area surrounding the target on a display of the imaging device and, using the display screen, select a reference point on the fluorescence image and a test point on the fluorescence image. In one example, the reference point may be spaced away from a clinical point of interest, such as a wound, and an area adjacent to or comprising part of the clinical area of interest, for example a wound, may be selected by the user as the test point. A processor of the imaging device will automatically calculate the temperature difference between these two regions. The device may then output an indication of the temperature difference to the display screen. This may include applying an overlay to a white light, fluorescence, and/or thermal image of the target and surrounding area. The device may also provide a thermal map of the imaged area, showing on the display screen a thermal map that identifies the temperature difference between the user selected reference point and the user selected test point. Examples of this type of map as well as other outputs including co-registered standard, fluorescence, and thermal images are shown in FIGS. 5A-12. In some embodiments, the output to the display of the device includes co-registered images captured by the device. In some embodiments, the output to the display includes the fluorescence image displayed in a side-by-side format with the indication of the temperature difference between the user selected reference point and the user selected test point. The indication of the temperature difference between the user selected reference point and the user selected test point can include an overlay on any one of the captured images, a thermal map, or other markings that indicate to a viewer the difference in temperature between the user selected reference point and the user selected test point. The indication may be conveyed through the use of color, markings, symbols, maps, and combinations thereof.

### EXAMPLES

As shown in the example of FIGS. 5A and 5B, an indication that the test point is warmer than the reference point may indicate the potential presence of inflammation or infection. In the example of FIGS. 5A and 5B, the positive temperature differential between the test point (a healed DFU) and the reference point, led to further investigation of the foot containing the healed DFU. This resulted in the discovery of an infection in the big toe of the “hot” foot. Additional swelling and signs of inflammation were found around the newly identified wound, consistent with the positive thermal signature shown in FIG. 5B. This, along with positive fluorescence (FL) signals (see center image of FIG. 5B which shows red fluorescence at the area bounded by the white line and identified by the white arrow), led to the patient being diagnosed with a deep tissue infection.

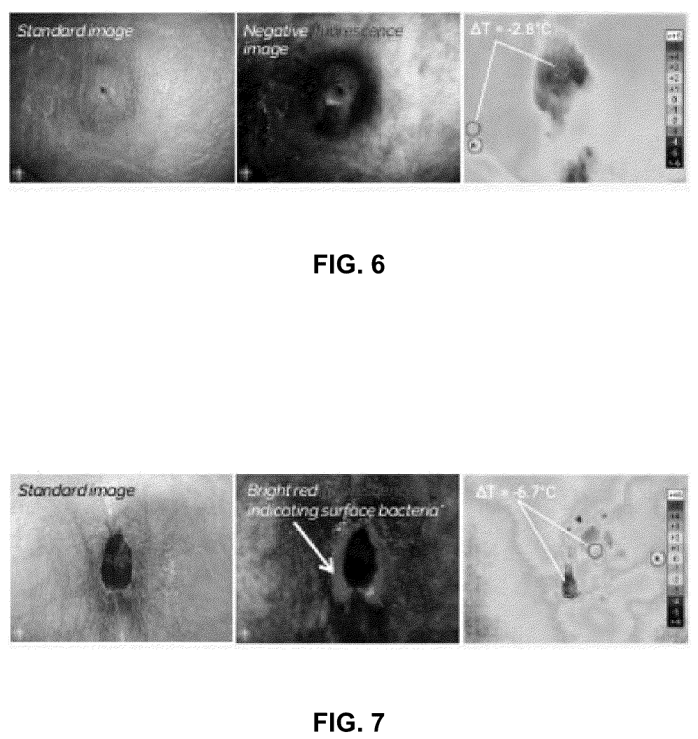

As shown in the example of FIG. 6, test points that are cooler than the reference point indicate the potential for decreased localised oxygen delivery, undermining, and devitalization. FIG. 6 shows three co-registed images, a standard image, a fluorescence image, and a thermal image. The fluoresence image was negative, ruling out a bacterial infection (later confirmed by swab). The thermal image highlighted a very cool region at the 9-12 o'clock position, with respect to the wound, that was 2.8° C. colder than the surrounding tissue. Clinical assessment confirmed extensive tunneling and undermining (up to 7 cm), indicating that the wound had grown since the previous assessment despite negative fluoresence findings and unremarkable clinical assessment.

In the example of FIG. 7, which shows from left to right a standard image of a wound, a fluorescence image of the wound, and a thermal image of the wound, the images being co-registered. In this example, the wound was a deep pilonidal cyst. FL imaging of the wound indicated a heavy bacterial burden (see the bright red fluorescence indicated by the white arrow in the center photo) at the wound periphery. Thermal imaging indicated cool regions (6.7° C. colder than the surrounding tissue) overlapping with the FL positive areas, suggesting poor perfusion and devitalization of bacteria-laden tissue at the periphery of the wound, which can impair healing.

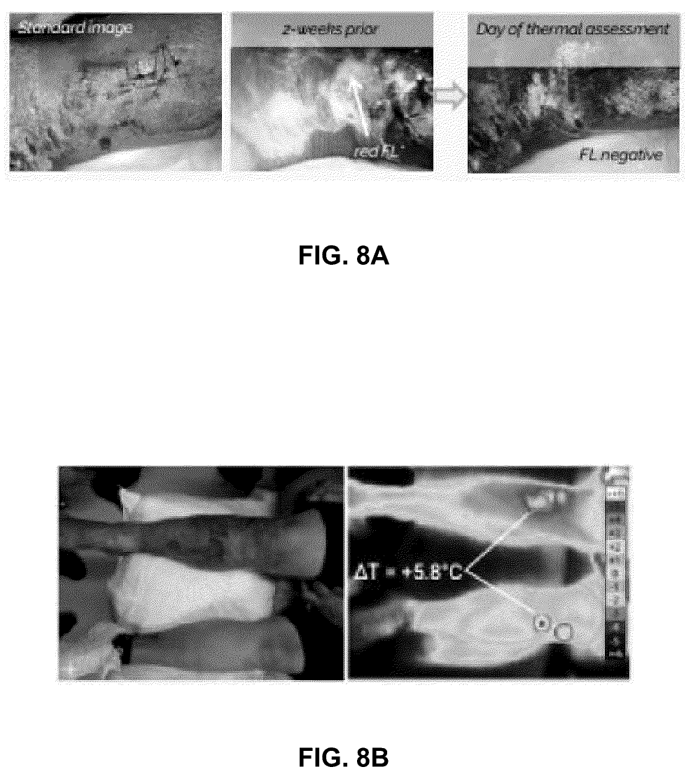

In the example of FIGS. 8A and 8B, a benefit of wound monitoring as taught by the present application is illustrated. FIG. 8A shows images that were taken two weeks prior to a thermal assessment. In the images of FIG. 8A, a standard image and a fluorescence image are shown. The initial FL image is positive for bacteria, showing red fluorescence in the FL (center) image as identified by the white arrow. Based on the positive fluorescence and in combination with clinical signs and symptoms, the patient was diagnosed with cellulitis. FL images were used to guide cleaning, debridement, and antimicrobial dressing selection to target surface-level bacterial loads and oral antibiotics were prescribed for any deeper cellulitis infection.

Two weeks later, on the last day of antibiotic treatment, a negative FL image (see third image in FIG. 8A) and swab confirmed the surface infection was resolved. However, thermal images taken that day (two weeks later) showed a positive temperature differential of 5.8° C. between the infected and contralateral limb (see FIG. 8B), indicating that the deep infection was still present, prompting the continuation of antibiotics.

The examples above were part of a 25-patient study in which wounds (diabetic and venous ulcers, surgical and post-traumatic wounds) were imaged using the MolecuLight DX with thermal module. The combination of the FL and thermal imaging had a positive impact on diagnosis and treatment planning, including diagnoses of infections including cellulitis and identification of unsuspected tunneling, undermining, and regions of poor perfusion. The combination of thermal and fluorescent imaging also facilitated simultaneous tracking of changes in surface level bacterial loads and deep tissue infection and enabled fluorescence-targeted debridement in regions of devitalized tissue. The data captured from co-registed thermal and fluorescence images are synergistic and complementary. This type of multi-modal imaging simultaneously identifieds elevated bacterial loads and regions of increased temperature associated with inflammation/infection, or decreased temperature associated with underming/poor perfusion.

Additional examples described below highlight further synergies between fluorescence and thermal imaging. These synergies may include the ability to identify issues underneath a patient's intact skin, such as sinus tracks, undermining, and abscesses. The use of these modalities together may also flag potential involvement of underlying bone and may also provide indications of healing.

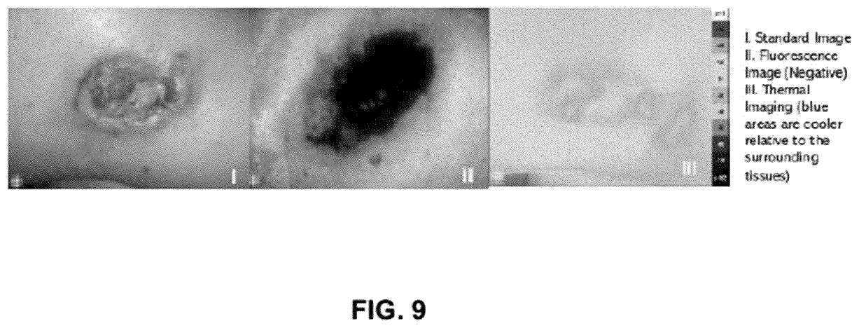

In the example of FIG. 9, which shows, from top to bottom, a standard image, a fluorescent image, and a thermal image, cooler temperatures surround the wound in areas covered by intact skin. These cooler temperatures suggest the presence of sinus tracks and undermining, which was confirmed through debridement and probing.

Similarly, although not shown in this set of images, self-contained areas of warmth that are wound adjacent and located under intact skin may suggest the presence of an abscess.

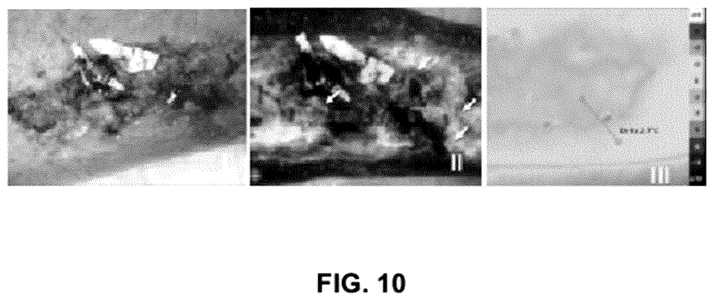

FIG. 10 shows, from top to bottom, a standard image, a fluorescence image, and a thermal image of infected, chronic post-radiation skin. The middle image shows faint blush fluorescence signals under scabs as identified by the white arrows. The thermal image shows blue (cooler) areas relative to the surrounding tissues. Cooler temperatures within infected wounds, particularly when proximal to a bone, may raise concern for bone involvement. This patient was sent for further imaging to rule out osteomyelitis.

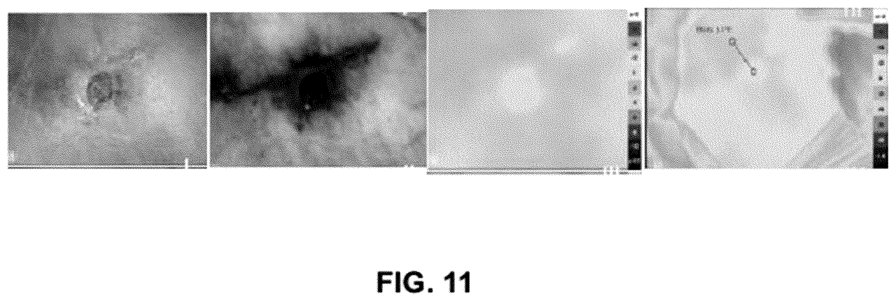

FIG. 11 shows, from top to bottom, a standard image, a fluorescence image, a close-up thermal image, and a distanced thermal image of a wound. The fluorescence image is negative-no bacterial fluorescence detected. Standard clinical assessment may be subjective but combining results from fluorescence and thermal imaging can mitigate concerns about complications. In the example shown, the wound is an abdominal surgical wound with granulation tissue and serous exudate. Thermal imaging reveals moderate to low relative temperatures with a uniform distribution. While homogenous warmth is typical in the abdomen, it does not necessarily exclude infection. However, negative fluorescence imaging suggest the absence of significant bacterial presence, aiding in ruling out infection.

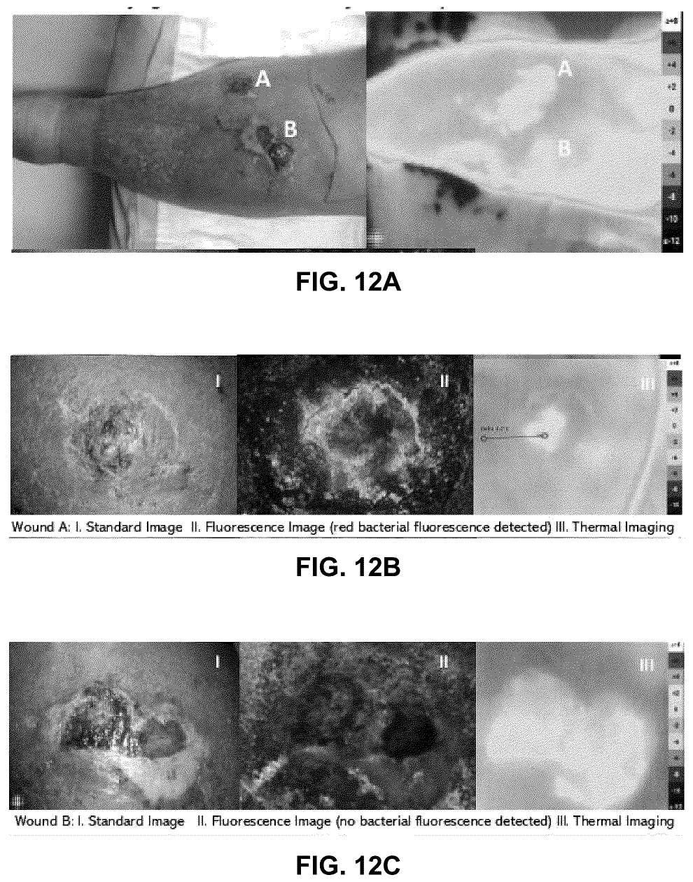

FIGS. 12A-12C show images of two wounds sustained in a motorcycle accident, two months post accident. FIG. 12A shows a standard image and a thermal image of both wounds. FIG. 12B shows a standard image, a fluorescence image, and a thermal image of wound A. FIG. 12C shows a standard image, a fluorescence image, and a thermal image of wound B. After multiple rounds of antibiotics the patient was referred to a specialized wound clinic where these images were taken. The thermal imaging showed elevated temperatures around the wounds, supporting visible indications of cellulitis. Fluorescence imaging located the bacterial infection's source in wound A, confirmed by fluorescence-guided sampling. Topical antibiotics were provided to supplement the systemic antibiotics and the wound healed.

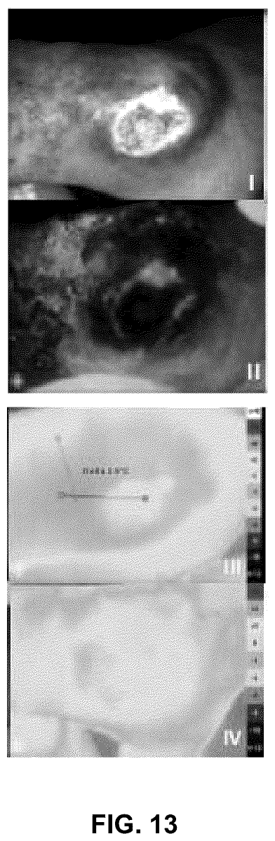

FIG. 13 shows, from top to bottom, a standard image, a fluorescence image, an up-close thermal image, and a distant thermal image of a pressure injury. Standard clinical inspection showed no signs of infection. However, the fluorescence imaging detected bright red fluorescence around the right lateral malleolar wound, indicating pathogenic bacterial loads. Thermal imaging confirmed increased temperatures in the same area, especially when captured from a further distance.

### Example Device

A device in accordance with the present disclosure includes the capability for fluorescence imaging and thermal imaging. The combination of fluorescence imaging and thermal imaging is synergystic, providing powerful diagnostic information to the clinician. Fluorescence imaging accurately and in real-time pinpoints bacterial presence including high bacterial loads or pathogenical bacterial loads. Thermal imaging offers insights into tissue inflammation and perfusion which also may provide diagnostic information relating to skin and soft tissue infection. Together, these modalities enhance wound diagnostic accuracy, from wound assessment to guiding targeted interventions and facilitates comprehensive wound assessment to improve patient outcomes. The integration of data from both fluorescence and thermal imaging offers a holistic understanding of the patient's condition.

One example of a wound monitoring device is a portable, handheld imaging system that includes an imaging device having two or more cameras (i.e., camera sensors) and a processor coupled to the imaging device for analyzing the images captured from the camera sensors to perform algorithms or other operations as will be described in more detail below. The imaging device, for example, includes a first, primary camera sensor and a second, secondary camera sensor. The first, primary camera sensor and the second, secondary camera sensor may be configured to capture standard, white light (WL) images, fluorescent (FL) images, near infrared (NIR) images, or infrared (IR) images. The sensors may be so configured by use with dedicated filters or filters selectable from a plurality of filters associated with the imaging device (e.g., filter wheel, tunable filters, etc.), in which the filters may be wavelength filters (e.g., low-pass filters, high-pass filters, band-pass filters, multi-band filters, etc.) and/or polarization filters. Thus, the method disclosed herein may be used to measure features captured in WL, FL, NIR, or IR images. In some implementations, to permit determination of the parallax value of a primary and secondary image (taken, respectively, by the primary and secondary camera sensors), the first camera sensor is separated from the second camera sensor by a predetermined, fixed separation distance. In other implementations, to permit depth measurement, a time-of-flight or other depth imaging sensor may be provided. The imaging device also includes a thermal module having a thermal image sensor to capture thermal images. In one example embodiment, the thermal camera may be a FLIR Lepton thermal imaging module. The thermal module may be permanently attached to the multi-modal imaging device or the thermal module may be detachably mounted on a housing of the multi-modal imaging device. In accordance with one aspect of the present teachings, the thermal module is mounted an angle on the housing of the imaging device, where the angle is selected such that a field of view (FOV) of the thermal module coincides with or is substantially the same as the field of view of the fluorescence image sensor/camera. The FOV of the thermal module may also coincide with or be substantially the same as the field of view of the white light camera. Providing mechanical alignment of the fields of view facilitates co-registration and co-localization of the thermal data, fluorescence data, and white light data.

In accordance with one aspect of the present teachings, a handheld portable device to examine skin and wounds in real-time is provided. The device instantly detects, visualizes, and analyzes bacteria and tissue composition. The device is a compact, handheld, device for noncontact and noninvasive imaging. It captures both white light (WL) and autofluorescence (AF) signals produced by tissue components and bacteria without the use of contrast agents. Although capable of detecting AF signals without use of contrast agents, one of ordinary skill in the art will understand that the devices disclosed herein can be used with contrast agents if desired. In addition to white light and fluorescence, the device also may capture thermal data from the imaged area. The device may be further configured to analyze the white light, fluorescence, and thermal data, correlate such data, and provide an output based on the correlation of the data, such as, for example, an indication of wound status, wound healing, wound infection, bacterial load, or other diagnostic information upon which an intervention strategy may be based. The device may co-register or co-localize the thermal data with fluorescence data and/or white light data. The fluorescence data provides an indication of bacterial presence, location, and bacterial load. The thermal information provides an indication of the presence of, for example, inflammation. Thus, when fluorescence data indicates the presence of bacteria and that data is co-registered/co-localized with thermal data indicating the presence of inflammation, an indication of the presence or potential presence of infection may be provided.

In accordance with one aspect of the present disclosure, the thermal module is accurate to within 0.5° C. and a temperature differential of 3° C. or more is indicative of an elevated temperature which may indicate infection.

The device may be configured to create and/or display composite images including green AF, produced by endogenous connective tissues (e.g., collagen, elastin) in skin, and red AF, produced by endogenous porphyrins in clinically relevant bacteria such as Staphylococcus aureus. Siderophores/pyoverdines in other species such as Pseudomonas aeruginosa appear blue-green in color with in vivo AF imaging. The device may provide visualization of bacterial presence, types, distribution, amounts in and around a wound as well as key information surrounding tissue composition (collagen, tissue viability, blood oxygen saturation). For example, the device may provide imaging of collagen composition in and around skin in real-time (via AF imaging).

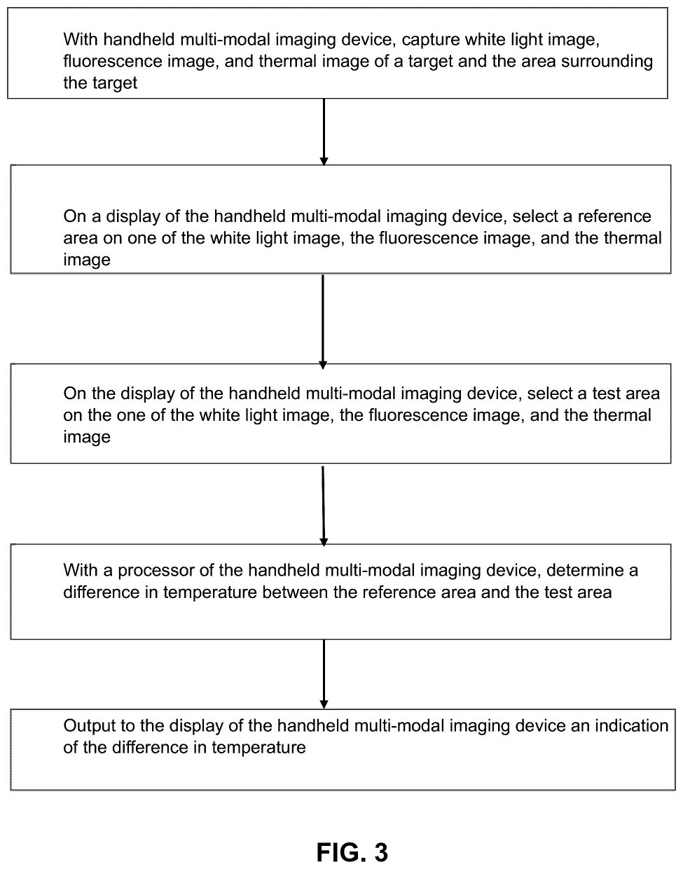

In accordance with one example embodiment of a method in accordance with the present disclosure, after a fluorescence image of the wound is captured on the imaging device, the user may select an area of interest on the FL image (which is displayed on a display screen of the imaging device). For example, an area of the FL image having a color indicative of a type of bacteria, such as an area of red, blue-green, or cyan, may be an area for further exploration and analysis. A user can select a first area of the FL image that does not contain the area of interest and set it as a reference point by tapping or circling the area on the touchscreen display of the device. The user can then select a test point in one of the areas of the FL image having a color indicative of bacteria by tapping or circling the area on the touchscreen display of the device. Once the reference point and test point are selected by the user, the processor of the device will determine the difference in temperature between the reference point and the test point. The processor will output an indication of the temperature difference to the display screen. An example method is shown in the flowchart of FIG. 3.

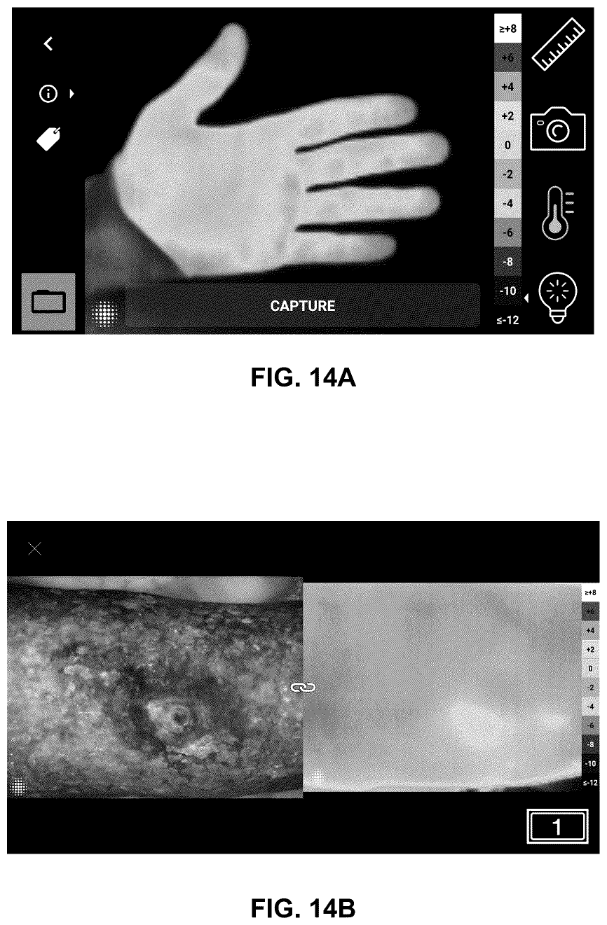

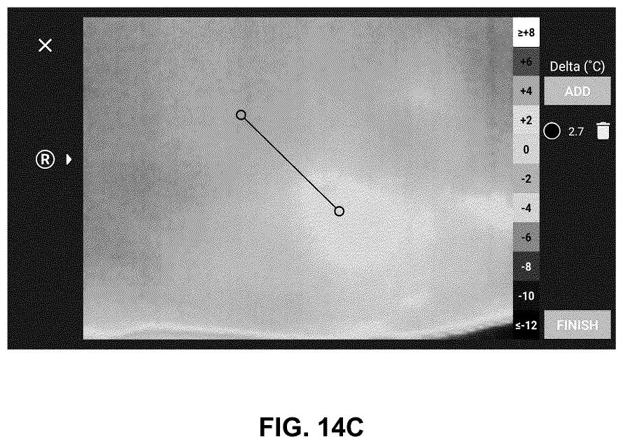

FIG. 14A shows an example user interface present on a display of the device for imaging. FIG. 14B shows a user interface present on a display of the device for review and FIG. 14C shows a user interface present on the display of the device for analysis. Note that the review screen shows a “link” between the two images, representing that the images are co-registered, with the FL and thermal images taken almost simultaneously.

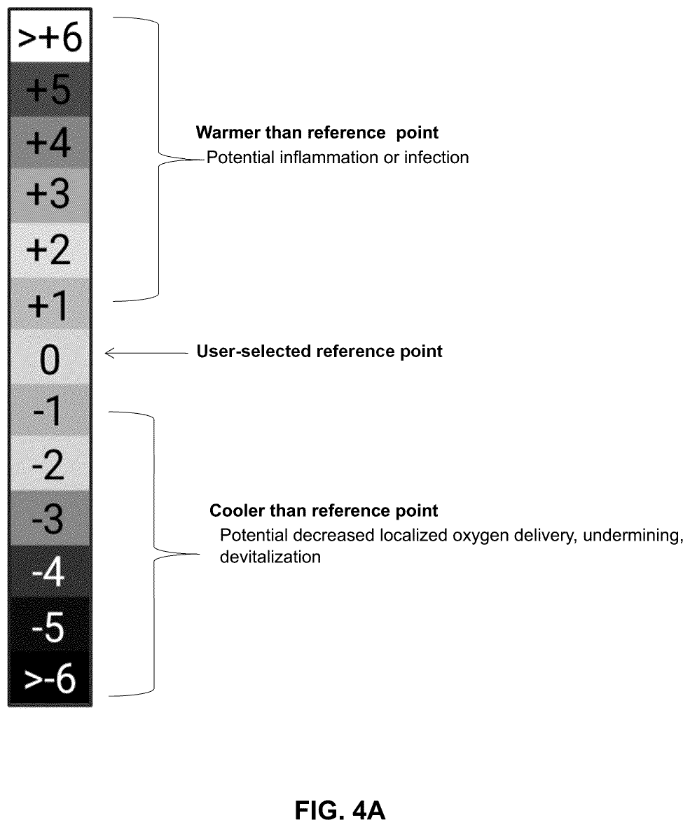

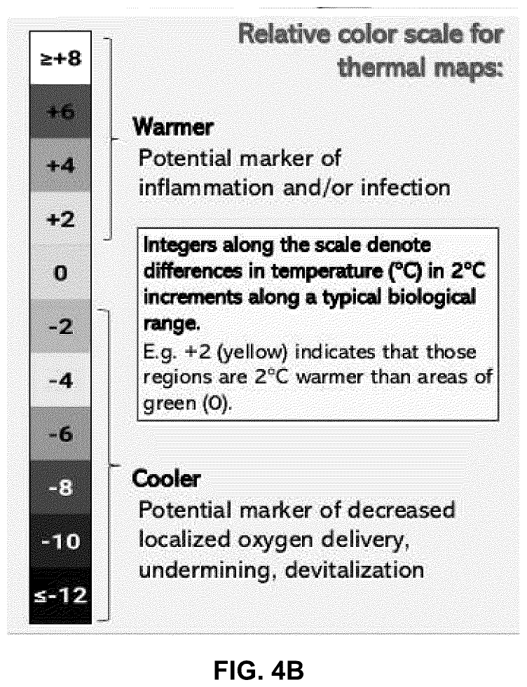

Example outputs are shown in FIGS. 5A-14C. The example outputs are shown as color images output by a multi-modal imaging device in accordance with the present disclosure, where the output is a thermal map of the imaged target and surrounding area, with a user selected reference point and user selected test point applied to the thermal map and a temperature differential between the two points being indicated numerically as well as by a relative color scale in which the user selected reference point has been set to zero on the scale and temperatures warmer (a positive temperature differential) and cooler (a negative temperature differential) than the reference point are shown in different colors by the number of degrees Celsius differentiating the test point relative to the reference point. Example relative color scales for the thermal map are shown in FIGS. 4A and 4B. In addition to the thermal map output, as shown in FIGS. 5A-12, the output may also include co-registered standard (i.e., white light) and fluorescence images.

In accordance with various exemplary embodiments of the present disclosure, the device may be configured to accurately detect and measure bacterial load in wounds in real-time, guide treatment decisions, and track wound healing over the course of antibacterial treatment. Additionally, bioluminescence imaging (BLI) may be used to correlate absolute bacterial load with FL signals obtained using the handheld device. The device may produce a uniform illumination field on a target area to allow for imagining/quantification of bacteria, collagen, tissue viability, and oxygen saturation.

In accordance with another aspect of the present disclosure, the device is configured to capture and generate images and videos that provide a map or other visual display of user selected parameters. Such maps or displays may correlate, overlay, co-register or otherwise coordinate data generated by the device based on input from one or more device sensors. Such sensors may include, for example, camera sensors configured to detect white light and/or fluorescent images and thermal sensors configured to detect heat signatures of a target. For example, the device may be configured to display color images, image maps, or other maps of user selected parameters such as, for example, bacteria location and/or biodistribution, collagen location, location and differentiation between live tissues and dead tissues, differentiation between bacterial species, location and extent of blood, bone, exudate, temperature and wound area/size. These maps or displays may be output by the device based on the received signals and may be produced on a single image with or without quantification displays. The user-selected parameters shown on the map may be correlated with one or more wound parameters, such as shape, size, topography, volume, depth, and area of the wound. For example, in accordance with one exemplary embodiment, it is possible to use a ‘pseudo-colored’ display of the fluorescence images/videos of wounds to color-code bacteria fluorescence (one color) and connective tissues (another color) etc. This may be accomplished by, for example, using a pixel-by-pixel coloring based on the relative amount of 405 nm light in the Blue channel of the resultant RGB image, green connective tissue fluorescence in the Green channel, and red bacteria fluorescence in Red channel. Additionally and/or alternatively, this may be accomplished by displaying the number of pixels in a given image for each of the blue, green and red channels which would represent amount of blood in tissue, amount of connective tissues, and amount of bacteria, respectively.

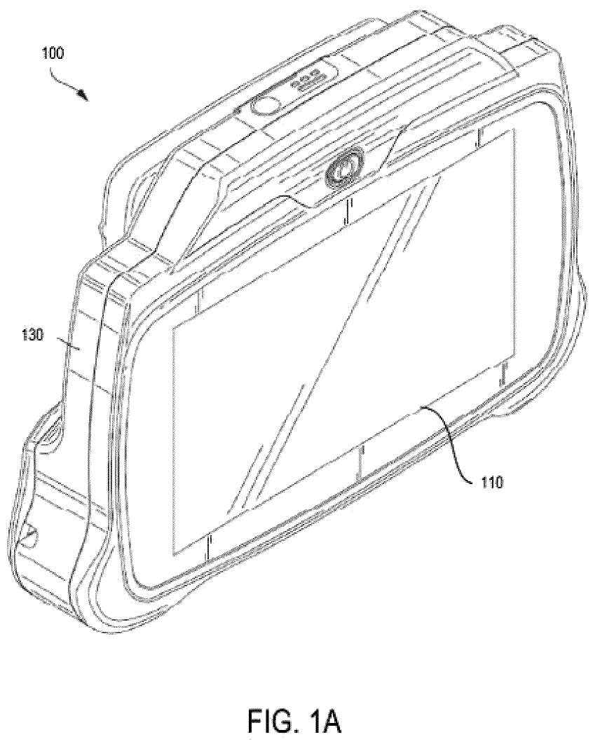

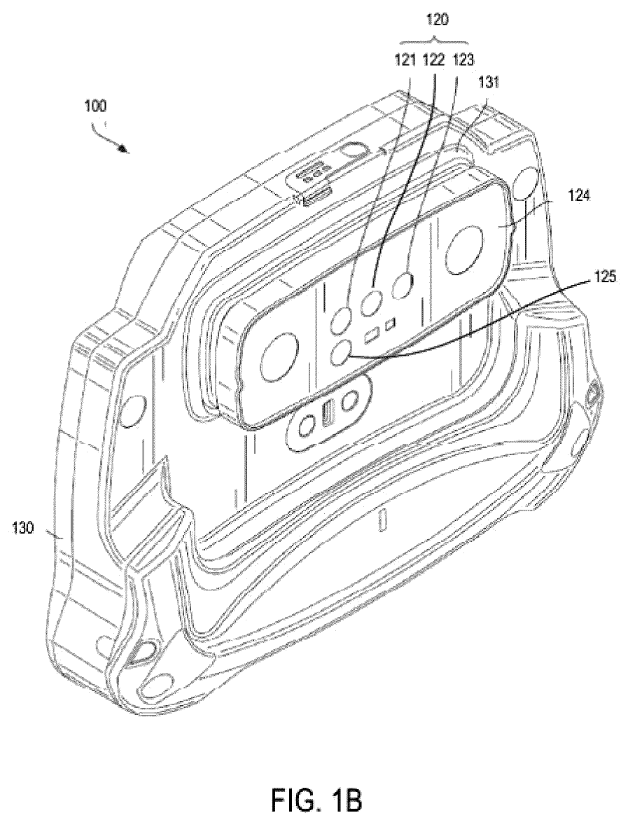

FIGS. 1A-B illustrate an exemplary wound monitoring device 100 in accordance with the present disclosure, in which FIG. 1A is a front perspective view of the wound monitoring device 100 and FIG. 1B is a rear perspective view of the wound monitoring device 100. Device 100 can be, for instance, the MolecuLight DX® device developed by MolecuLight®. Device 100 is non-contact and no imaging contrast agents are required for white light and/or fluorescence imaging. As illustrated, the wound monitoring device 100 includes a display device 110, an imaging device 120, and a device housing 130.

The imaging device 120 includes at least one image sensor, such as, for example, a first image sensor 121, a second image sensor 122, and a third image sensor 123. Each of the first image sensor 121, the second image sensor 122, and the third image sensor 123 may individually be implemented as image sensors that may be used for one or more of WL, FL, IR, and thermal imaging. In one example, the first image sensor 121 and the third image sensor 123 are together configured for stereoscopic white-light imaging, and the second image sensor 122 is configured for fluorescence imaging. In another example, the first image sensor 121 and the third image sensor 123 are together configured for stereoscopic fluorescence imaging, and the second image sensor 122 is configured for white-light imaging. In yet another example, the first image sensor 121 is configured for white-light imaging, the second image sensor 122 is configured for fluorescence imaging of a first wavelength or wavelength range, and the third image sensor 123 is configured for fluorescence imaging of a second wavelength or wavelength range. The physical arrangement (i.e., ordering) of the first image sensor 121, the second image sensor 122, and the third image sensor 123 may also be different from that shown in FIG. 1B. Although for ease of illustration the wound monitoring device 100 depicts three image sensors, the present disclosure includes an imaging device 120 including any number of image sensors so long as at least one image sensor capable of receiving fluorescence signals is present.

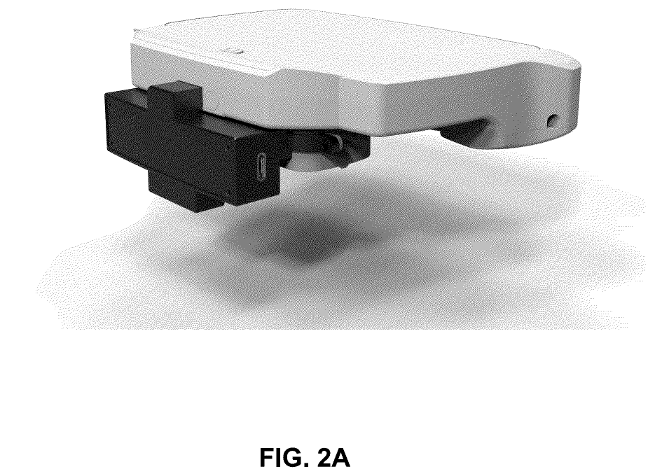

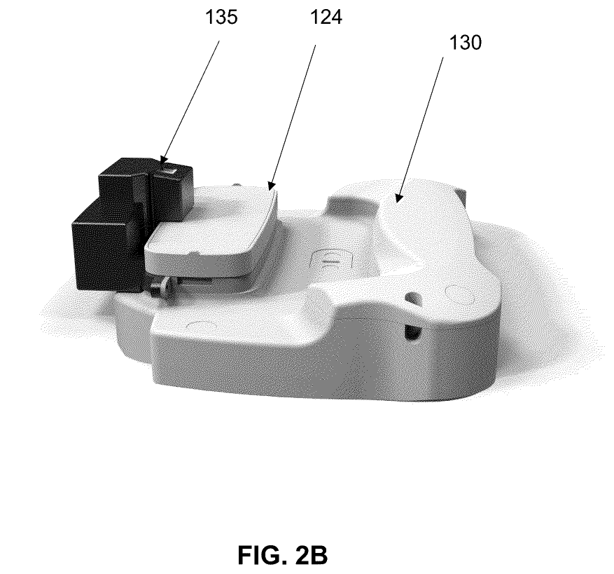

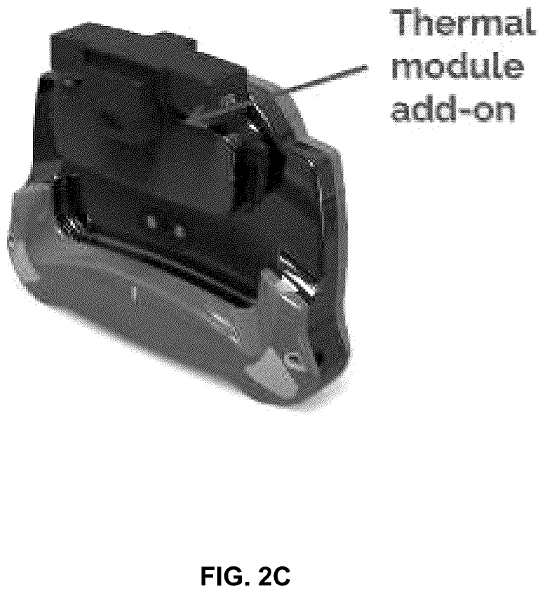

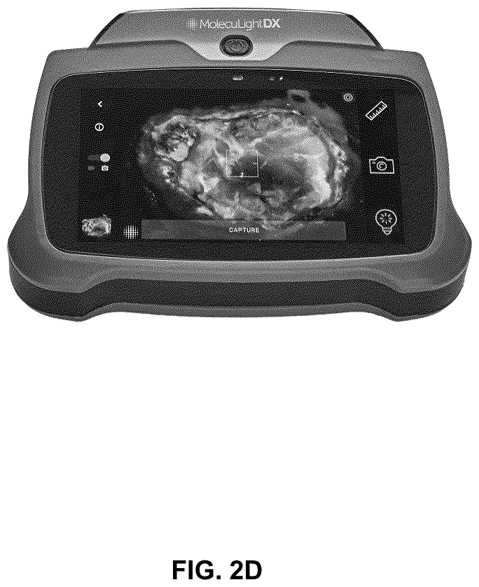

In the illustration of FIG. 2, the first image sensor 121, the second image sensor 122, and the third image sensor 123 are arranged in an optical housing 124. The optical housing 124 may further include one or more excitation light sources, such as excitation light source 125 implemented as a light-emitting diode (LED). The optical housing 124 may additionally include at least one white light torch, rangefinder, temperature sensor, etc.

The device housing 130 may include a physical user interface, such as a power button, one or more input buttons, and so on. The device housing 130 may also include various input and output ports, such as wired charging ports, inductive charging ports, universal serial bus (USB) ports, and/or other peripheral ports. As shown in FIG. 1B, the device housing 130 includes an optical mount 131 on which the imaging device 120 is mounted. The optical mount 131 may provide for a permanent physical mount or may provide for a removable mount in implementations where the imaging device 120 is modular (i.e., may be replaced with other imaging devices such as endoscope heads). The device housing 130 may be of a unitary construction or may include multiple housing components attached together. While not particularly illustrated in FIGS. 1A-B, the device housing 130 may include an internal space in which various components such as a processor (e.g., a central processing unit (CPU)), a memory, a program storage, and the like are disposed. In some example embodiments, a memory may store data, for example in a look up table, related to various conditions associated with temperature differentials calculated by the processor and the positivity or negativity of the fluorescence determination (e.g., positive fluorescence corresponds to the presence of bacteria and negative fluorescence corresponds to the absence of bacteria). The processor may cause indications of the presence or absence of various conditions associated with a calculated (or determined) temperature differential to be displayed on the display screen of the device.

For example, in response to a determination that the temperature of the test area is higher than the temperature of the reference area by 3° C. or more, the processor may output an indication of the presence of a bacterial infection. In another example, in response to a determination that the temperature of the test area is lower than the temperature of the reference area by 3° C. or more, the processor may output an indication of one or more of bacterial infection negative, possible tunneling or undermining, and poor perfusion. The various potential outcomes associated with combinations of positive or negative fluorescence and positive or negative temperature differentials may be associated with indications of the presence or absence of various conditions as highlighted in the examples described above.

As shown in FIGS. 2A-2C, the imaging device may further include a thermal imaging module 135. The thermal imaging module 135 may be permanently mounted on the housing 130 or otherwise form a permanent part of the imaging device 120. Alternatively, the thermal imaging module 135 may be detachably mounted on the optical housing 124 of the imaging device 120. An example of a thermal imaging module 135 that may be used with the imaging device is a FLIR Lepton thermal imaging module.

Illustrative examples of systems, methods, and devices described herein are provided below. An embodiment of a system, method, and/or device described herein may include any one or more, and any combination of, the clauses described below:

Clause 1. A method of analyzing a wound, comprising, with a handheld multi-modal imaging device, capturing a fluorescence image of a target and a surrounding area, and capturing a thermal image of the target and the surrounding area; on a display of the handheld multi-modal imaging device: selecting a reference area on one of the fluorescence image and the thermal image, and selecting a test area on the one of the fluorescence image and the thermal image; and with a processor of the multi-modal imaging device: determining a difference in temperature between the reference area and the test area, and outputting to the display an indication of the difference in temperature, wherein the indication of the difference in temperature is displayed side by side with the fluorescence image.

Clause 2. The method of Clause 1, wherein outputting to the display an indication of the difference in temperature comprises applying an overlay to the thermal image of the target and the surrounding area.

Clause 3. The method of Clause 2, wherein the overlay includes a first mark identifying the control area and a second mark identifying the test area.

Clause 4. The method of any one of Clauses 1-3, further comprising applying a label indicative of the difference in temperature on the thermal image and wherein outputting to the display the indication of the difference in temperature comprises displaying the labeled thermal image side by side with the fluorescence image.

Clause 5. The method of any one of Clauses1-4, further comprising setting a temperature of the reference area to zero, wherein outputting the indication of the difference in temperature includes outputting an indication of whether the test area is warmer or cooler than the reference area.

Clause 6. The method of any one of Clauses 1-5, further comprising creating a thermal map of the target and the surrounding area using a relative color scale, wherein a temperature of zero on the relative color scale corresponds to the temperature of the control area.

Clause 7. The method of any one of Clauses 1-6, further comprising, with a handheld multi-modal imaging device, capturing a white light image of the target and the surrounding area; and, wherein, selecting a reference area on one of the fluorescence image and the thermal image, includes selecting a reference area on one of the white light image, the fluorescence image, and the thermal image; and wherein selecting a test area on the one of the fluorescence image and the thermal image includes selecting a test area on one of the white light image, the fluorescence image, and the thermal image.

Clause 8. The method of any one of Clauses 1-7, further comprising co-registering, co-localizing, and/or overlaying the white light image, the fluorescence image, and the thermal image.

Clause 9. The method of any one of Clauses 1-8, wherein selecting a test area on the one of the white light image, the fluorescence image, or the thermal image comprises selecting an area on the fluorescence image as the test area.

Clause 10. The method of Clause 9, further comprising, in response to a determination that the temperature of the test area is higher than the temperature of the reference area by 3° C. or more, outputting an indication of the presence of a bacterial infection.

Clause 11. The method of Clause 9, further comprising, in response to a determination that the temperature of the test area is lower than the temperature of the reference area by 3° C. or more, outputting an indication of one or more of bacterial infection negative, possible tunneling or undermining, and poor perfusion.

Clause 12. The method of any one of Clauses 1-11, wherein the target is a wound in tissue.

Clause 13. A multi-modal imaging device, comprising: a housing; a white light camera configured to capture white light images; a fluorescence camera configured to capture fluorescence images; a thermal imaging module; a processor configured to receive white light image data, fluorescence image data, and thermal image data; and a display, wherein the display is configured to allow a user to select a reference area and a test area on one or more of a white light image, a fluorescent image, and a thermal image, and wherein the processor is further configured to determine a difference in temperature between the reference area and the test area, and output to the display an indication of the difference in temperature and a fluorescence image containing the reference area and the test area.

Clause 14. The device of Clause 13, wherein a field of view of the white light camera is substantially the same as a field of view of the thermal imaging module.

Clause 15. The device of Clause 13 or Clause 14, wherein a field of view

of the fluorescent camera is substantially the same as a field of view of the thermal imaging module.

Clause 16. The device of any one of Clauses 13-15, wherein a field of view of the white light camera is substantially the same as a field of view of the fluorescent camera.

Clause 17. The device of any one of Clauses 13-16, wherein the white light camera is a stereoscopic camera.

Clause 18. The device of any one of Clauses 13-17, wherein the white light camera is adjacent to the fluorescent camera on the housing in a first direction.

Clause 19. The device of Clause 18, wherein the thermal imaging module is adjacent to the white light camera and fluorescent camera on the housing in a second direction orthogonal to the first direction.

Clause 20. The device of any one of Clauses 13-19, wherein the thermal imaging module is movable between a first position, in which a field of view of the thermal imaging module substantially coincides with a field of view of the fluorescent camera and a field of view of the white light camera, and a second position, in which a field of view of the thermal imaging module does not substantially coincide with a field of view of the fluorescent camera or a field of view of the white light camera.

Clause 21. The device of any one of Clauses 13-20, wherein the thermal imaging module is removably attached to the housing.

Clause 22. The device of any one of Clauses 13-21, wherein the processor is configured to apply an overlay indicating the difference in temperature to the thermal image of the target and the surrounding area.

Clause 23. The device of Clause 22, wherein the overlay includes a first mark identifying the reference area and a second mark identifying the test area.

Clause 24. The device of any one of Clauses 13-23, wherein processor is configured to apply a label indicative of the difference in temperature on the thermal image.

Clause 25. The device of any one of Clauses 13-24, wherein the processor is further configured to set a temperature of the reference area to zero, and wherein outputting the indication of the difference in temperature includes outputting an indication of whether the test area is warmer or cooler than the reference area.

Clause 26. The device of any one of Clauses 13-25, wherein the processor is further configured to create a thermal map of the target and the surrounding area using a relative color scale, wherein a temperature of zero on the relative color scale corresponds to the temperature of the control area.

Clause 27. The device of any one of Clauses 13-26, wherein the processor is configured to co-register, co-localize, and/or overlay the white light image data, the fluorescent image data, and the thermal image data.

Clause 28. The device of any one of Clauses 13-27, wherein the thermal module includes a thermal sensor configured: to receive thermal radiation from the target and the surrounding area and to output an electrical signal representing the receive thermal radiation; and an accessory housing configured for removable attachment to the housing of the handheld multi-modal imaging device.

Clause 29. The device of Clause 28, wherein the accessory housing includes a surface feature configured to receive an attachment for a surgical drape.

Clause 30. The device of any of Clauses 13-29, wherein the target is a wound in tissue.

Clause 31. The device of any one of Clauses 13-26 and 28-30, wherein the indication of the difference in temperature is a thermal map or a thermal image and the thermal map or thermal image is co-registered and displayed side-by-side with the fluorescence image.

Clause 32. The device of Clause 31, wherein the processor is configured to co-register, co-localize, and/or overlay the white light image data, the fluorescent image data, and the thermal image data.

Clause 33. The device of Clause 32, wherein the processor is configured to display the co-registered, co-localized, and/or overlaid image data.

Clause 34. A method of analyzing a wound, comprising with a handheld multi-modal imaging device capturing a first image of a target and a surrounding area, and capturing a second image of the target and the surrounding area, wherein the second image is a fluorescent image; co-registering the first image and the second image; receiving a first input from a user of the handheld multi-modal imaging device, the first input identifying a reference point on the target and the surrounding area; receiving a second input from the user of the handheld multi-modal imaging device, the second input identifying a test point on the target and the surrounding area; with a processor of the multi-modal imaging device, determining a difference in temperature between the reference point and the test point based on a thermal measurement of the target and surrounding area; and on a display of the multi-modal imaging device, displaying the co-registered images with an indication of the difference in temperature.

Clause 35. A multi-modal imaging device, comprising an optical sensor configured to detect fluorescence from a target and a surrounding area; a thermal imaging module configured to capture a thermal image of the target and the surrounding area; a display; and a processor configured to: co-register a fluorescence image and a thermal image, receive a first input from a user of the device, the first input identifying a reference point on the target and the surrounding area, receive a second input from the user of the device, the second input identifying a test point on the target and the surrounding area; determine a difference in temperature between the reference point and the test point; and output an indication of the difference in temperature to the display.

Clause 36. A multi-modal imaging device, comprising a first optical sensor configured to detect fluorescence from a target and a surrounding area; a stereoscopic white light camera configured to capture white light images of the target and the surrounding area; a thermal imaging module configured to capture a thermal image of the target and the surrounding area; a touchscreen display; and a processor, the processor configured to co-register a white light image, a fluorescence image, and a thermal image, receive a first input from a user of the device, the first input identifying a reference point on the target and the surrounding area, receive a second input from the user of the device, the second input identifying a test point on the target and the surrounding area; determine a difference in temperature between the reference point and the test point; and output to the display the co-registered fluorescence and thermal images, wherein one of the fluorescence and thermal images includes an indication of the difference in temperature.

Clause 37. A multi-modal imaging device, comprising a housing; a fluorescence camera configured to capture fluorescence images; a thermal imaging module; a processor configured to receive fluorescence image data and thermal image data; and a touchscreen display, wherein the display is configured to allow a user to select a reference area and a test area on a captured image, and wherein the processor is further configured to determine a difference in temperature between the user-selected reference area and the user-selected test area, and output to the display a fluorescence image containing the reference area and the test area and an image co-registered with the fluorescence image and containing an indication of the difference in temperature between the user-selected reference area and the user-selected test area.

Clause 38. A method of analyzing a wound, comprising capturing a fluorescence image of a target and a surrounding area with a handheld multi-modal imaging device, and capturing a thermal image of the target and the surrounding area with the handheld multi-modal imaging device; selecting via a touchscreen display of the handheld multi-modal imaging device, on one of the captured images, a reference area and a test area; determining, with a processor of the multi-modal imaging device, a difference in temperature between the reference area and the test area; and displaying on the touchscreen display of the handheld multi-modal imaging device, the fluorescence image and the thermal image, and an indication of the difference in temperature between the reference area and the test area.

Clause 39. The method of Clause 38, wherein displaying, on the touchscreen display of the handheld multi-modal imaging device, the fluorescence image, and the thermal image, and an indication of the difference in temperature between the reference area and the test area comprises applying an overlay to the thermal image of the target and the surrounding area.

Clause 40. The method of Clause 39, wherein the overlay includes a first mark identifying the reference area and a second mark identifying the test area.

Clause 41. The method of any one of Clauses 38-40, further comprising applying a label indicative of the difference in temperature on the thermal image and displaying, on the touchscreen display of the handheld multi-modal imaging device the indication of the difference in temperature comprises displaying the labeled thermal image side by side with the fluorescence image.

Clause 42. The method of any one of Clauses 38-41, further comprising setting a temperature of the reference area to zero, wherein displaying, on the touchscreen display of the handheld multi-modal imaging device, the indication of the difference in temperature includes displaying an indication of whether the test area is warmer or cooler than the reference area.

Clause 43. The method of any one of Clauses 38-42, further comprising creating a thermal map of the target and the surrounding area using a relative color scale, wherein a temperature of zero on the relative color scale corresponds to the temperature of the reference area.

Clause 44. The method of any one of Clauses 38-43, further comprising, in response to a determination that the temperature of the test area is higher than the temperature of the reference area by 3° C. or more, outputting an indication of the presence of a bacterial infection.

Clause 45. The method of any one of Clauses 38-43, further comprising, in response to a determination that the temperature of the test area is lower than the temperature of the reference area by 3° C. or more, outputting an indication of one or more of bacterial infection negative, possible tunneling or undermining, and poor perfusion.

Clause 46. The method of any one of Clauses 38-45, wherein the target is a wound in tissue.

The above description and associated figures teach the best mode of the disclosed devices, systems, and methods, and are intended to be illustrative and not restrictive. Many embodiments and applications other than the examples provided would be apparent to those skilled in the art upon reading the above description. The scope should be determined, not with reference to the above description, but instead with reference to the appended claims, along with the full scope of equivalents to which such claims are entitled. It is anticipated and intended that future developments will occur in the technologies discussed herein, and that the disclosed systems and methods will be incorporated into future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary is made herein. In particular, the use of the singular articles such as “a,” “the,” “said,” etc. should be read to recite one or more of the indicated elements unless a claim recites an explicit limitation to the contrary.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method of analyzing a wound, comprising:
with a handheld multi-modal imaging device:
capturing a fluorescence image of a target and a surrounding area, and
capturing a thermal image of the target and the surrounding area;
on a display of the handheld multi-modal imaging device:
selecting a reference area on one of the fluorescence image and the thermal image, and
selecting a test area on the one of the fluorescence image and the thermal image; and
with a processor of the multi-modal imaging device:
determining a difference in temperature between the reference area and the test area, and
outputting to the display an indication of the difference in temperature,
wherein the indication of the difference in temperature is displayed side by side with the fluorescence image.

2. The method of claim 1, wherein outputting to the display an indication of the difference in temperature comprises applying an overlay to the thermal image of the target and the surrounding area.

3. The method of claim 2, wherein the overlay includes a first mark identifying the control area and a second mark identifying the test area.

4. The method of claim 1, further comprising applying a label indicative of the difference in temperature on the thermal image and wherein outputting to the display the indication of the difference in temperature comprises displaying the labeled thermal image side by side with the fluorescence image.

5. The method of claim 1, further comprising setting a temperature of the reference area to zero, wherein outputting the indication of the difference in temperature includes outputting an indication of whether the test area is warmer or cooler than the reference area.

6. The method of claim 1, further comprising creating a thermal map of the target and the surrounding area using a relative color scale, wherein a temperature of zero on the relative color scale corresponds to the temperature of the control area.

7. The method of claim 1, further comprising:
with a handheld multi-modal imaging device capturing a white light image of the target and the surrounding area; and
wherein:
selecting a reference area on one of the fluorescence image and the thermal image, includes selecting a reference area on one of the white light image, the fluorescence image, and the thermal image; and
selecting a test area on the one of the fluorescence image and the thermal image includes selecting a test area on one of the white light image, the fluorescence image, and the thermal image.

8. The method of claim 7, further comprising co-registering, co-localizing, and/or overlaying the white light image, the fluorescence image, and the thermal image.

9. The method of claim 8, wherein selecting a test area on the one of the white light image, the fluorescence image, or the thermal image comprises selecting an area on the fluorescence image as the test area.

10. The method of claim 9, further comprising, in response to a determination that the temperature of the test area is higher than the temperature of the reference area by 3° C. or more, outputting an indication of the presence of a bacterial infection.

11. The method of claim 9, further comprising, in response to a determination that the temperature of the test area is lower than the temperature of the reference area by 3° C. or more, outputting an indication of one or more of bacterial infection negative, possible tunneling or undermining, and poor perfusion.

12. The method of claim 1, wherein the target is a wound in tissue.

13. A multi-modal imaging device, comprising:
a housing;
a white light camera configured to capture white light images;
a fluorescence camera configured to capture fluorescence images;
a thermal imaging module;
a processor configured to receive white light image data, fluorescence image data, and thermal image data; and
a display,
wherein the display is configured to allow a user to select a reference area and a test area on one or more of a white light image, a fluorescent image, and a thermal image, and
wherein the processor is further configured to:
determine a difference in temperature between the reference area and the test area, and
output to the display an indication of the difference in temperature and a fluorescence image containing the reference area and the test area.

14. The device of claim 13, wherein a field of view of the white light camera is substantially the same as a field of view of the thermal imaging module.

15. The device of claim 13, wherein a field of view of the fluorescent camera is substantially the same as a field of view of the thermal imaging module.

16. The device of claim 13, wherein a field of view of the white light camera is substantially the same as a field of view of the fluorescent camera.

17. The device of claim 13, wherein the white light camera is a stereoscopic camera.

18. The device of claim 13, wherein the white light camera is adjacent to the fluorescent camera on the housing in a first direction.

19. The device of claim 18, wherein the thermal imaging module is adjacent to the white light camera and fluorescent camera on the housing in a second direction orthogonal to the first direction.

20. The device of claim 13, wherein the thermal imaging module is movable between a first position, in which a field of view of the thermal imaging module substantially coincides with a field of view of the fluorescent camera and a field of view of the white light camera, and a second position, in which a field of view of the thermal imaging module does not substantially coincide with a field of view of the fluorescent camera or a field of view of the white light camera.

21. The device of claim 13, wherein the thermal imaging module is removably attached to the housing.

22. The device of claim 13, wherein the processor is configured to apply an overlay indicating the difference in temperature to the thermal image of the target and the surrounding area.

23. The device of claim 22, wherein the overlay includes a first mark identifying the reference area and a second mark identifying the test area.

24. The device of claim 13, wherein processor is configured to apply a label indicative of the difference in temperature on the thermal image.

25. The device of claim 13, wherein the processor is further configured to set a temperature of the reference area to zero, and wherein outputting the indication of the difference in temperature includes outputting an indication of whether the test area is warmer or cooler than the reference area.

26. The device of claim 13, wherein the processor is further configured to create a thermal map of the target and the surrounding area using a relative color scale, wherein a temperature of zero on the relative color scale corresponds to the temperature of the control area.

27. The device of claim 13, wherein the processor is configured to co-register, co-localize, and/or overlay the white light image data, the fluorescent image data, and the thermal image data.

28. The device of claim 13, wherein the thermal module includes:
a thermal sensor configured to receive thermal radiation from the target and the surrounding area and to output an electrical signal representing the receive thermal radiation; and
an accessory housing configured for removable attachment to the housing of the handheld multi-modal imaging device.

29. The device of claim 28, wherein the accessory housing includes a surface feature configured to receive an attachment for a surgical drape.

30. The device of claim 13, wherein the target is a wound in tissue.

31. The device of claim 13, wherein the indication of the difference in temperature is a thermal map or a thermal image and the thermal map or thermal image is co-registered and displayed side-by-side with the fluorescence image.

32. The device of claim 31, wherein the processor is configured to co-register, co-localize, and/or overlay the white light image data, the fluorescent image data, and the thermal image data.

33. The device of claim 32, wherein the processor is configured to display the co-registered, co-localized, and/or overlaid image data.

34. A method of analyzing a wound, comprising:
with a handheld multi-modal imaging device:
capturing a first image of a target and a surrounding area, and
capturing a second image of the target and the surrounding area, wherein the second image is a fluorescent image;
co-registering the first image and the second image;
receiving a first input from a user of the handheld multi-modal imaging device, the first input identifying a reference point on the target and the surrounding area;
receiving a second input from the user of the handheld multi-modal imaging device, the second input identifying a test point on the target and the surrounding area;
with a processor of the multi-modal imaging device, determining a difference in temperature between the reference point and the test point based on a thermal measurement of the target and surrounding area; and
on a display of the multi-modal imaging device, displaying the co-registered images with an indication of the difference in temperature.

35. A multi-modal imaging device, comprising:
an optical sensor configured to detect fluorescence from a target and a surrounding area;
a thermal imaging module configured to capture a thermal image of the target and the surrounding area;
a display; and
a processor configured to:
co-register a fluorescence image and a thermal image,
receive a first input from a user of the device, the first input identifying a reference point on the target and the surrounding area,
receive a second input from the user of the device, the second input identifying a test point on the target and the surrounding area;
determine a difference in temperature between the reference point and the test point; and
output an indication of the difference in temperature to the display.

36. A multi-modal imaging device, comprising:
a first optical sensor configured to detect fluorescence from a target and a surrounding area;
a stereoscopic white light camera configured to capture white light images of the target and the surrounding area;
a thermal imaging module configured to capture a thermal image of the target and the surrounding area;
a touchscreen display; and
a processor configured to:
co-register a white light image, a fluorescence image, and a thermal image,
receive a first input from a user of the device, the first input identifying a reference point on the target and the surrounding area,
receive a second input from the user of the device, the second input identifying a test point on the target and the surrounding area;
determine a difference in temperature between the reference point and the test point; and
output to the display the co-registered fluorescence and thermal images, wherein one of the fluorescence and thermal images includes an indication of the difference in temperature.

37. A multi-modal imaging device, comprising:
a housing;
a fluorescence camera configured to capture fluorescence images;
a thermal imaging module;
a processor configured to receive fluorescence image data and thermal image data; and
a touchscreen display,
wherein the display is configured to allow a user to select a reference area and a test area on a captured image, and
wherein the processor is further configured to:
determine a difference in temperature between the user-selected reference area and the user-selected test area, and
output to the display a fluorescence image containing the reference area and the test area and an image co-registered with the fluorescence image and containing an indication of the difference in temperature between the user-selected reference area and the user-selected test area.

38. A method of analyzing a wound, comprising:
capturing a fluorescence image of a target and a surrounding area with a handheld multi-modal imaging device, and
capturing a thermal image of the target and the surrounding area with the handheld multi-modal imaging device;
selecting via a touchscreen display of the handheld multi-modal imaging device, on one of the captured images, a reference area and a test area;
determining, with a processor of the multi-modal imaging device, a difference in temperature between the reference area and the test area; and
displaying, on the touchscreen display of the handheld multi-modal imaging device, the fluorescence image, and the thermal image, and an indication of the difference in temperature between the reference area and the test area.

39. The method of claim 38, wherein displaying, on the touchscreen display of the handheld multi-modal imaging device, the fluorescence image, and the thermal image, and an indication of the difference in temperature between the reference area and the test area comprises applying an overlay to the thermal image of the target and the surrounding area.

40. The method of claim 39, wherein the overlay includes a first mark identifying the reference area and a second mark identifying the test area.

41. The method of claim 38, further comprising applying a label indicative of the difference in temperature on the thermal image and displaying, on the touchscreen display of the handheld multi-modal imaging device the indication of the difference in temperature comprises displaying the labeled thermal image side by side with the fluorescence image.

42. The method of claim 38, further comprising setting a temperature of the reference area to zero, wherein displaying, on the touchscreen display of the handheld multi-modal imaging device, the indication of the difference in temperature includes displaying an indication of whether the test area is warmer or cooler than the reference area.

43. The method of claim 38, further comprising creating a thermal map of the target and the surrounding area using a relative color scale, wherein a temperature of zero on the relative color scale corresponds to the temperature of the reference area.

44. The method of claim 38, further comprising, in response to a determination that the temperature of the test area is higher than the temperature of the reference area by 3° C. or more, outputting an indication of the presence of a bacterial infection.

45. The method of claim 38, further comprising, in response to a determination that the temperature of the test area is lower than the temperature of the reference area by 3° C. or more, outputting an indication of one or more of bacterial infection negative, possible tunneling or undermining, and poor perfusion.

46. The method of claim 38, wherein the target is a wound in tissue.
